(19) United States
(12) Patent Application Publication
(10) Pub. No.: US 2020/0338139 A1
(43) Pub. Date: Oct. 29, 2020

(54) COMPOSITIONS FOR MICROBIAL ANTI-ADHESION

(71) Applicants: Muhammed Majeed, Edison, NJ (US); Kalyanam Nagabhushanam, East Windsor, NJ (US); Anurag Pande, East Windsor, NJ (US); Shaheen Majeed, Springville, UT (US)

(72) Inventors: Muhammed Majeed, Edison, NJ (US); Kalyanam Nagabhushanam, East Windsor, NJ (US); Anurag Pande, East Windsor, NJ (US); Shaheen Majeed, Springville, UT (US)

(73) Assignee: SAMI LABS LIMITED, BANGALORE (IN)

(21) Appl. No.: 16/765,372

(22) PCT Filed: Dec. 4, 2018
(86) PCT No.: PCT/US18/63728

§ 371 Date: May 19, 2020

Related U.S. Application Data
(60) Provisional application No. 62/594,072, filed on Dec. 4, 2017.

(51) Int. Cl. A61K 35/742; A61K 36/45; A61K 9/00

(52) U.S. Cl. CPC A61K 35/742; A61K 9/0014; A61K 9/0053; A61K 36/45

(57) ABSTRACT

The present invention discloses the microbial anti-adhesion effect of probiotic bacteria Bacillus coagulans MTCC 5856. More specifically the invention discloses the ability of a composition containing probiotic bacteria Bacillus coagulans MTCC 5856 in inhibiting the adhesion of harmful pathogenic microbes to the skin and mucosal surfaces thereby preventing the occurrence of an infection. Compositions containing probiotic bacteria Bacillus coagulans MTCC 5856 along with plant/fruit extracts for use as an anti-adhesion agent are also disclosed.

### CROSS REFERENCE TO RELATED APPLICATIONS

This is a national phase application of PCT application no. PCT/US18/63728, filed on 4 Dec. 2018, claiming priority of U.S. provisional application No. U.S. 62/594,072 filed on 4 Dec. 2017.

### BACKGROUND OF THE INVENTION

### Field of Invention

The invention in general relates to probiotics. More specifically the invention relates to microbial anti-adhesion property of compositions containing probiotic bacterium Bacillus coagulans.

### Description of Prior Art

The skin and the mucosal linings such as mouth, lining of the gut, nasal passages, airways, urinary tract and genitals are prone to microbial infections. Bacteria and Fungi which infect the mucosal surfaces generally include, but not limited to, E. coli, Candida albicans, Pneumococci, Staphylococci, Streptococci, Chlamydia Trachomatis, Treponema Pallidum, Haemophilus Ducreyi, and Tinea Cruris. Viruses like Herpes SimplexVirus and Human Papiloma Virus also infect the skin and mucosal surfaces leading to inflammation and discomfort. The list of infections of the skin and mucosa are described in the following prior art documents.
Eversole L R, Inflammatory diseases of the mucous membranes, Part 1. Viral and fungal infections. J Calif Dent Assoc. 1994;22(4):52-7.
Marini A, Hengge U R. Important viral and bacterial infections of the skin and mucous membrane, Internist (Berl). 2009;50(2):160-70.
Morey L, Genital and mucous membrane lesions, Infectious Diseases, Infectious diseases advisor, https://www.infectiousdiseaseadvisor.com/infectious-diseases/genital-and-mucous-membrane-lesions/article/609384/, accessed 26 Nov. 2018.
Marques S A, Fungal infections of the mucous membrane, Dermatol Ther. 2010; 23(3):243-50.
Thrush and mucosal injection, LIFE—Leading International Fungal Education, http://www.life-worldwide.org/fungal-diseases/mucosal-infection/, accessed 28 Nov. 2018.
Djojodimedjo T et al., Escherichia coli infection induces mucosal damage and expression of proteins promoting urinary stone formation, Urolithiasis. 2013;41(4):295-301.
Krishnan P A, Fungal infections of the oral mucosa, Indian Journal of Dental Research, 2012;23(5):650-659.
Dahlén G, Bacterial infections of the oral mucosa, Periodontology, 2009;49:13-38.

The general means for treating skin and mucosal infections include administration of antibiotics, anti-bacterial, anti-viral and anti-fungal agents. Due to the presence of severe side effects, more safe, non-toxic, economical and effective ways of treating these infections are now developed, which involve administration of probiotic organisms. Probiotic organisms like Bacillus sp., Lactobacillus sp. and Bifidobacteria have been reported to possess anti-microbial effects (WO 98/47374). U.S. Pat. No. 9,226,943 and publication no. US20160082052 disclose the microbial anti-adhesion property of Lactobacillus johnsonii. It is well known in the scientific art that biological effects of probiotics are strain specific and effect produced by one strain/species cannot to generalised to all probiotic strains/species, as evidenced in
A. Guidelines for the evaluation of probiotics in food, joint FAO/WHO Working Group Report on Drafting Guidelines for the Evaluation of Probiotics in Food, London, Ontario, Canada, April 30 and May 1, 2002, See section 3.1 indicating that “The current state of evidence suggests that probiotic effects are strain specific. Strain identity is important to link a strain to a specific health effect as well as to enable accurate surveillance and epidemiological studies.”:
B. Probiotics: In Depth/NCCIH, U.S. Department of Health and Human Services (http://www.hhs.gov/) National Institutes of Health (http://www.nih.gov/); and
C. Indian Council of Medical Research/Department of Biotechnology, Ministry of Science and Technology, Government of India, New Delhi, ICMR-DBT GUIDELINES FOR EVALUATION OF PROBIOTICS IN FOOD, 2011, Section 2, Subsection 2.3)

Hence, there still exists an unmet industrial need to find a superior probiotic strain that acts as an effective anti-microbial agent, specifically an anti-adhesion agent. U.S. Pat. No. 9,717,766, US 20160058805 and WO 2016/033572 disclose the anti-microbial effect of Bacillus coagulans MTCC 5856 by inhibiting growth of gram negative bacteria, but do not disclose the anti-adhesion property of the probiotic organism. The present invention discloses a novel and nonobvious microbial anti-adhesion effect of probiotic bacteria Bacillus coagulans MTCC 5856, which prevents the pathogenic microbes from binding to the mucosal membranes, thereby preventing the infection from occurring in the first place. Plant extracts, specifically fruit extracts and powders, are also reported to exhibit excellent anti-adhesion properly (Howell et al., A-type cranberry proanthocyanidins and uropathogenic bacterial anti-adhesion activity, Phytochemistry. 2005;66(18):2281-91). The present invention also discloses the anti-adhesion effect of a composition containing Bacillus coagulans and plant extracts.

It is the principle objective of the invention is to disclose the microbial anti-adhesion effect of probiotic bacteria Bacillus coagulans MTCC 5856.

It is another objective of the invention to disclose the management and prevention of infectious of mucosal surfaces using a composition containing probiotic bacteria Bacillus coagulans MTCC 5856.

It is yet another objective of the invention to disclose a composition containing probiotic bacteria Bacillus coagulans MTCC 5856 and plant/fruit extracts for use as an anti-adhesion agent.

The present invention solves the above mentioned objectives and provides further related advantages.

### DEPOSIT OF BIOLOGICAL MATERIAL

The deposit of biological material Bacillus coagulans SBC37-01 bearing accession number MTCC 5856, mentioned in the instant application has been made on 19 Sep. 2013 at Microbial Type Culture Collection & Gene Bank (MTCC), CSIR-Institute of Microbial Technology, Sector 39-A, Chandigarh 160036, India.

### SUMMARY OF THE INVENTION

The present invention discloses the microbial anti-adhesion effect of probiotic bacteria Bacillus coagulans MTCC 5856. The invention specifically discloses the ability of a composition containing probiotic bacteria Bacillus coagulans MTCC 5856 in inhibiting the adhesion of harmful pathogenic microbes to the mucosal surfaces thereby preventing the occurrence of an infection. A composition containing probiotic bacteria Bacillus coagulans MTCC 5856 along with plant/fruit extracts for use as an anti-adhesion agent is also disclosed.

Other features and advantages of the present invention wall become apparent from the following more detailed description, taken in conjunction with the accompanying images, which illustrate, by way of example, the principle of the invention.

### BRIEF DESCRIPTION OF DRAWINGS

FIG. 1 is a graphical representation showing the percentage Urinary Anti-adhesion activity of a composition containing Bacillus coagulans MTCC 5856 and cranberry fruit extracts over a period of 0-48 hours.

FIG. 2 is a graphical representation showing the urinary bacterial anti-adhesion activity of the study subjects administered with a composition containing Bacillus coagulans MTCC 5856 and cranberry fruit extracts.

### DESCRIPTION OF PREFERRED EMBODIMENTS

The present invention discloses a method for inhibiting the adhesion of pathogenic micro-organisms to skin and mucosal surfaces of a mammal, said method comprising steps of administering a composition comprising of probiotic micro-organism Bacillus coagulans to said mammal to bring about an inhibitory effect on microbial adhesion. In a related embodiment, the mucosal surfaces are selected from the group consisting of but not limited to, gastrointestinal tract, urinogenital tract, oral mucosa, and respiratory tract. In another related embodiment, the composition further contains a plant extract or powder. In another related embodiment, the plant extract or powder is prepared from whole fruit, seeds or juice. In another related embodiment, the micro-organism Bacillus coagulans strain is preferably Bacillus coagulans MTCC 5856.

The present invention also discloses a method of therapeutic management and prevention of microbial infections of the skin and mucosal surfaces said method comprising steps of administering a composition comprising probiotic micro-organism Bacillus coagulans to mammals in need of such therapeutic management. In a related embodiment, the management and prevention of infectious of mucosal surfaces is brought about by inhibiting the adhesion of the pathogenic microbe to the mucosal surface. In a related embodiment, the mucosal surfaces are selected from the group consisting of but not limited to, gastrointestinal tract, urinogenital tract, oral mucosa, and respiratory tract. In another related embodiment, die composition further contains a plant extract or a powder in another related embodiment, the plant extract or powder is prepared from whole fruit, seeds or juice. In another related embodiment, the micro-organism Bacillus coagulans strain is preferably Bacillus coagulans MTCC 5856.

In another preferred embodiment the invention discloses a composition comprising probiotic micro-organism Bacillus coagulans and a plant extract or powder for use as an anti-adhesion agent. In another related embodiment, the plant powder or extract is prepared from whole frail, seeds or juice In another related embodiment, the micro-organism Bacillus coagulans strain is preferably Bacillus coagulans MTCC 5856. In a related embodiment, the plant is preferably Cranberry. In yet another related embodiment, the cranberry species is selected from the group consisting of Vaccinium oxycoccos, Vaccinium microcarpum, Vaccinium macrocarpon, and Vaccinium erythrocarpum. In yet another related embodiment, the cranberry extract/plant powder in the composition does not inhibit the probiotic effect of Bacillus coagulans. In yet another related embodiment the micro-organism Bacillus coagulans does not alter the therapeutic effect of cranberry extract/plant powder. In yet another related embodiment the composition is formulated with pharmaceutically/nutraceutically acceptable excipients, adjuvants, bases, diluents, carriers, conditioning agents, bioavailability enhancer, antioxidants and preservatives and administered orally in form of tablets, capsules, syrups, gummies, powders, suspensions, emulsions, chewables, candies and eatables. In yet another related embodiment the composition is formulated with pharmaceutically/cosmeceutically acceptable excipients, adjuvants, bases, diluents, carriers, conditioning agents, bioavailability enhancers, antioxidants and preservatives and/or incorporated into formulations containing skin care ingredients and administered topically in the form of creams, gels, lotions, powder, serum, oil, suspensions and ointments.

The specific examples included herein below illustrate the aforesaid most preferred embodiments of the present invention.

### EXAMPLE 1

### In Vitro Bacterial Anti-Adhesion Activity

The in vitro bacterial anti-adhesion activity (AAA) on a per weight basis, of Bacillus coagulans MTCC 5856 was evaluated in comparison with Cranberry extract/plant powder per se and a composition containing Bacillus coagulans MTCC 5856 and Cranberry extract/plant powder.

Samples were suspended (60 mg/ml) in PBS, neutralized with 1 N NaOH, diluted serially (2-fold), and tested for bacterial anti-adhesion activity utilizing an HRBC hemagglutination assay specific for uropathogenic P-fimbriated E. coli according to Foo et al. (Phytochemistry, 54(2), 173-81, 2000). The concentration at which hemagglutination activity was suppressed by 50% was recorded as the endpoint for the assay and was considered the minimum inhibitory concentration (MIC). The lower the MIC, the higher the anti-adhesion activity (AAA) of the sample. Anti-adhesion assays were repeated three times and the results averaged The standard deviation for the assay is +/− one dilution on each side of the MIC. Anti-adhesion assays were repeated three times and the results averaged. Controls included wells containing bacteria+PBS, HRBC+PBS, bacteria+test compound, HRBC+test compound, and bacteria+HRBC. The results are tabulated in Table 1:

TABLE 1
in vitro bacterial anti-adhesion activity

| | | | AAA Whole |
|---|---|---|---|
| | | | Product |
| Sample | Product | Ref. | (mg/mL) |
| 1 | Cranberry extract/ | 0.100 grams | 30-60 |
| | plant powder + Probiotic | | |
| | B coagulans | | |
| | MTCC 5856 Blend | | |
| 2 | Cranberry Seed Extract/ | 0.050 grams | 60 |
| | plant powder (60 mesh) | | |
| 3 | B coagulans | 0.050 grams | Negative at 60 |
| | MTCC 5856 15 Billion | | |
| 4 | Cranberry Juice | 0.050 grams | 30 |

The final concentration at which anti-adhesion activity could be detected was recorded above. The smaller the AAA number, the greater the activity. The dilution series begin at 60 mg/mL which is not expected to have a biologically relevant effect on adhesion. Average anti-adhesion activity for whole cranberry powders we have tested is about 3.8-30 mg/mL, with a few powders having exceptional activity at 0.2-0.4 mg/mL.

### EXAMPLE 2

### Bacterial Anti-Adhesion Activity in Human Urine: Cranberry Juice Powder Plus Probiotic Bacillus coagulans MTCC 5856

Methods: Pre-Visit Subject Preparation:

Participant inclusion and exclusion criteria: 10 women end 10 men, healthy, between the ages of 25 and 60, no current urinary infections, no diabetes, or antibiotic use for 6 months.

Dietary restrictions: participants refrained from consuming all cranberry, blueberry, pomegranate, grape, chocolate and other high-flavonoid products for a 3-day wash out period prior to consuming test products and throughout testing period.

Treatment Product Administration and Urine Collection:

A background urine sample was collected clean-catch at time 0 prior to consumption of treatment capsule on day 1. One 500-mg dose of treatment capsule (Cranberry Juice Powder (Fruit d'Or Nutraceuticals as one example) Plus Probiotics (Bacillus coagulans MTCX 3856) was administered to 20 participants tn the evening on day 1 and again in the morning of day 2.

On day 2, urine was collected at 6, 12, 24, 36 and 48 hours following product consumption in the morning. On urine collection days, additional fluid consumption was standardized to avoid dilution of urine samples and allow for detection of anti-adhesion activity, if present. Urine samples were centrifuged, filtered (0.45 micron filter) and immediately frozen at −20 C.

Bacterial Anti-Adhesion Testing of Urines:

Thawed urines were tested full strength for bacterial anti-adhesion activity utilizing a mannose-resistant human red blood cell (HRBC) hemagglutination assay specific for uropathogenic P-fimbriated E. coli according to Foo et al. (Phytochemistry, 54(2), 173-81, 2000) and Howell et al., (Phytochemistry, 66(18):2281-91, 2005). A 30-μL drop of each urine was incubated with 10 μL of bacterial suspension on a 24-well polystyrene plate for 10 min at room temperature on a rotary shaker. Freshly drawn HRBCs (Al, Rh+) were suspended (3%) in PBS and added separately (1-μL drops) to test suspensions, which were then incubated for 20 min on a rotary shaker at room temperature and evaluated microscopically for the ability to prevent agglutination.

Anti-adhesion activity of each urine sample was scored visually based on a quantitative estimation of percent agglutination of each sample using the following scale: 0=no anti-adhesion activity. 1=50% anti-adhesion activity, 2=100% anti-adhesion activity. A score of 2 indicates significant anti-adhesion activity in the urine, whereas a score of 1 indicates moderate activity. The detection limits of the anti-adhesion assay are not high, enough to allow quantification of the activity in each urine sample via a dilution series, therefore the result is presented as either a positive or a negative for the activity of each sample. Anti-adhesion assays were repeated four times per sample and the results averaged. Controls included wells containing bacteria+PBS, HRBC+PBS, bacteria+test material, HRBC+test material, and bacteria+HRBC.

Adverse Events:

A follow-up interview administered a week after the study was conducted to screen participants for potential adverse events resulting from the treatment.

Results:

Changes in Urine pH:

Urinary pH averaged 6.21, eliminating a bacteriostatic effect. Cranberry consumption has historically not resulted in decreases in bacterial growth, as the urinary pH must be reduced to 5.5 or lower. The 500-mg dose of Cranberry Juice Powder (Fruit d'Or Nutraceuticals) Plus Probiotics (Bacillus coagulans MTCC 5856) administered BID for the one-day test period did not elicit a significant decrease in urinary pH sufficient to cause a decrease in bacterial growth.

Changes in Bacterial Anti-Adhesion Activity:

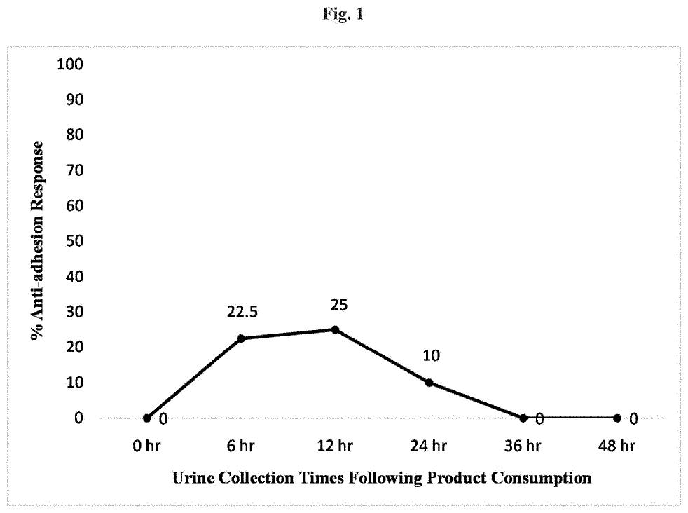

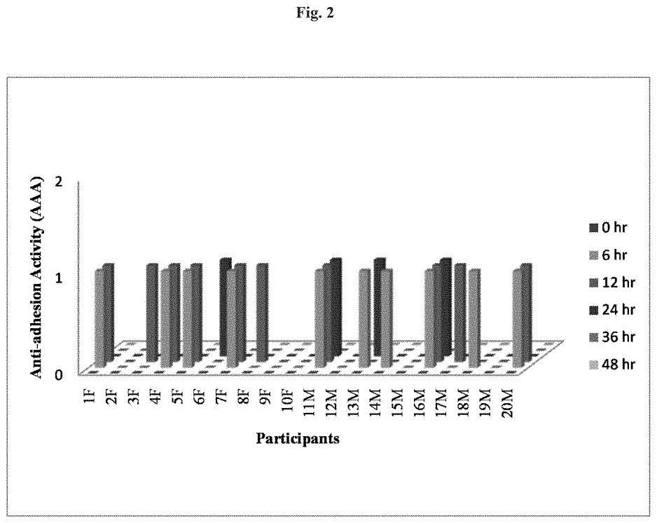

The changes in Bacterial Anti-adhesion Activity in depicted in FIG. 1 and FIG. 2. No anti-adhesion activity was detected in urines prior to product consumption (Time 0). Overall, 75% of the participants elicited some response to the cranberry/probiotic treatment. The percentage of observed urinary anti-adhesion activity recorded for all participants over every time period post-ingestion (from 6-48 hours yielded 12% overall response to the product (24 out of a possible 200). The product yielded a 25% response at 12 hours, which was the peak activity period. The pharmacokinetic activity increased gradually to 6 hours (22.5% response), maintained activity at 12 hours but then decreased rapidly after 24 hours (10% response). Of the women, 75% tested elicited a response in at least one of the time periods with an overall response of 11%, while 80% of the men responded in at least one time period with an overall response of 13%. These data are based on soluble proanthocyanidins (PACs) that can be measured and tested in die bioassays utilized in this study. However, there may be PACs in the treatment product that may be high molecular weight and insoluble in aqueous solvents.

Adverse Events:

Interviews conducted with the 20 participants found no adverse reactions or negative comments from ingesting the treatment product.

The composition has further advantages.

The cranberry extract/plant powder in the composition does not inhibit the probiotic effect of Bacillus coagulans. Similarly, the micro-organism Bacillus coagulans does not alter the therapeutic effect of cranberry extract/plant powder, thereby exhibiting a symbiotic relationship. Further the plant power/extract have many fibers which elicit many therapeutic benefits when administered along with Bacillus coagulans MTCC 5856 which is previously disclosed in U.S. Pat. No. 9,717,766, US 20160058805 and WO 2016/033572. The composition works synergistically to maintain urinary tract, vaginal, digestive, oral and immune health.

### EXAMPLE 3

### Formulations Containing Bacillus coagulans MTCC 5856

The composition containing Bacillus coagulans MTCC 5856 can be formulated with pharmaceutically/nutraceutically acceptable excipients, adjuvants, bases, diluents, carriers, conditioning agents, bioavailability enhancers, antioxidants and preservatives and administered orally in form of tablets, capsules, syrups, gummies, powders, suspensions, emulsions, chewables, candies and eatables. Further it can also be formulated with pharmaceutically/cosmeceutically acceptable excipients, adjuvants, bases, diluents, carriers, conditioning agents, bioavailability enhancers, antioxidants and preservatives and/or incorporated into formulations containing skin care ingredients and administered topically in the form of creams, gels, lotions, powder, serum, oil, suspensions and ointments.

In a related aspect, one or more anti-oxidants and anti-inflammatory agents are selected from the group consisting of, but not limited to, vitamin A, D, E, K, C, B complex, rosmarinic acid. Alpha Lipoic Acid, oxyresveratrol, Ellagic Acid, Glycyrrhizinic Acid, Epigallocatechin Gallate, plant polyphenols, Glabridin, moringa oil, oleanolic acid, Oleuropein, Camosic acid, urocanic acid, phytoene, lipoid acid, lipoamide, ferritin, desferal, billirubin, billiverdin, melanins, ubiquinone, ubiquinol, ascorbyl palmitate, Mg ascorbyl phosphate, ascorbyl acetate, tocopherols and derivatives such as vitamin E acetate, uric acid, u-glucosylrutin, calalase and the superoxide dismutase, glutathione, selenium compounds, butylated hydroxyanisole (BHA), butylated hydroxytoluene (BHT), sodium metabisulfite (SMB), propyl gailate (PG) and amino acid cysteine.

In another related aspect, one or more bioavailability enhancers are selected from the group, but not limited to, pipeline, tetrahydropiperine, quercetin, Garlic extract, ginger extract, and naringin.

In another related aspect, one or more skin care ingredients are selected from the group consisting of, but not limited to, Alpha Lipoic Acid, oxyresveratrol, Beet root extract, Boswellia serrata Extract, β boswellic acids, Boswellia serrata oil, Centelia asiatica Extract, triterpenes, Garcinia indica extract, anthocyanins, Cocos nucifera extract and juice, Coleus forskohlii Extract, forskolin, Coleus forskohlii Oil, Tetrahydropiperine, Ellagic Acid, Gallnut Extract, polyphenols, Galanga Extract, Glycyrrhizinic Acid, Green Tea Extract, Epigallocatechin Gallate, Licorice extract, MonoAmmonium Glycyrrhizinate, Limonoids, Oleanolic Acid, Cosmetic peptides (Oleanolic acid linked to Lvs-Thr-Thr-Lys-Ser, Oleanolic acid linked to Lys-Val-Lys), Oleuropein. Piper longumine extinct, piperine, Ellagic acid, Pomegranate Extract (Water Soluble), pterostilbene, resveratrol, Pterocarpus santalinus extract. Rosemary Extract, Rosmarinic Acid, Amla extract, beta glucogallin, tetrahydrocurcumin, Salvia Officinalis (Sage) Leaf Extract, Ursolic Acids, Saponins, Sesamum indicum (Sesame) Seed Extract, Sesamin and sesamolin, moringa oil, moringa seed extract, Horse Chestnut Extract, Vitex Oil, Xymenynic Acid, ethyl ascorbic acid, Argan oil, Lemon peel extract, turmeric oil, Barley Beta Glucans, coenzyme Q10, olive oil, avocado oil and cranberry oil.

Tables 2-5 provide illustrative examples of formulations containing Bacillus coagulans MTCC 5856 (LACTOSPORE) suitable for maintaining oral, gastrointestinal and urinogenital health.

TABLE 2
Bacillus coagulans Tablet

Active Ingredients
Bacillus coagulans MTCC 5856: 2 billion cfu
Excipients
Microcrystalline cellulose, Colloidal silicon dioxide, Magnesium stearate

TABLE 3
Bacillus coagulans Capsule

Active Ingredients
Bacillus coagulans MTCC 5856: 2 billion cfu
Excipients
Maltodextrin

TABLE 4
Bacillus coagulans Drink mix

Active Ingredients
Bacillus coagulans MTCC 5856: 2 billion cfu
Cranberry extract/fibers
Excipients
Maltodextrin, Taurine, Citric acid, Sucralose,
Flavouring agent, Vitamin B6 and Vitamin B12
Directions: Add 5 g of premix to 200 ml cold water and stir

TABLE 5
Bacillus coagulans oral wash

Active Ingredients
Bacillus coagulans MTCC 5856: 2 billion cfu
Cranberry fruit powder
Excipients
Essential oils, Flavouring agents, Emulsifiers, Preservatives

Tables 6-7 provide illustrative examples of skin care formulations containing Bacillus coagulans MTCC 5856 (commercially available as LACTOSPORE)

TABLE 6
Skin care Cream

Active Ingredients
Bacillus coagulans 100 to 2 billion cfu
Amaranthus extract, Niacinamide, Vitamin E,
Shea butter, Olive oil, D-Panthenol, Cranberry extract
Other ingredients/Excipients
Bioavailability enhancers (Piperine extract or
Tetrahydropiperine (Cosmoperine ®)), Fragrance,
Thickeners (Cellulose derivatives or
Acrylates Cross Polymer), Emulsifiers,
Preservatives (Sabilize ®), pH modifiers,
Chelating agents, Emollients and other solvents

TABLE 7
Skin care Ointment

Active Ingredients
Bacillus coagulant 100 to 2 billion, cfu
Cranberry Powder
Other ingredients/Excipients
Petroleum Base, Bioavailability enhancers (Piperine extract or
Tetrahydropiperine (Cosmoperine ®)),
Preservatives, Fragrance, Thickners and
emulsifiers, Chelating agents, antioxidatns

The above formulations are merely illustrative examples; any formulation containing the above active ingredient intended for the said purpose will be considered equivalent.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention. The scope of the invention is to be interpreted only in conjunction with the appended claims.

We claim:

1. A method for inhibiting the adhesion of pathogenic micro-organisms to the skin and mucosal surfaces of a mammal, said method comprising steps of administering a composition comprising of probiotic micro-organism Bacillus coagulans to said mammal to bring about an inhibitory effect on microbial adhesion.

2. The method as in claim 1, wherein the mucosal surfaces are selected from the group consisting of, gastrointestinal tract, urinogenital tract, oral mucosa, and respiratory tract.

3. The method as in claim 1, wherein the composition further contains a plant extract or powder.

4. The method as in claim 1, wherein the plant extract or powder is prepared from whole fruit, seeds or juice.

5. The method as in claim 1, wherein the micro-organism Bacillus coagulans strain is preferably Bacillus coagulans MTCC 5856.

6. A method of therapeutic management and prevention of microbial infections of the skin and mucosal surfaces said method comprising steps of administering orally a composition comprising probiotic micro-organism Bacillus coagulans to mammals in need of such therapeutic management.

7. The method as in claim 6, wherein the management and prevention of infections of mucosal surfaces is brought about by inhibiting the adhesion of the pathogenic microbe to the mucosal surface.

8. The method as in claim 6, wherein the mucosal surfaces are selected from the group consisting of but not limited to, gastrointestinal tract, urinogenital tract, oral mucosa, and respiratory tract.

9. The method as in claim 6, wherein the composition further contains a plant extract or powder.

10. The method as in claim 6, wherein the plant extract or powder is prepared from whole fruit, seeds or juice.

11. The method as in claim 6, wherein the micro-organism Bacillus coagulans strain is preferably Bacillus coagulans MTCC 5856.

12. A composition comprising probiotic micro-organism Bacillus coagulans and a plant extract or powder for use as an anti-adhesion agent.

13. The composition as in claim 12, wherein the plant powder or extract is prepared from whole fruit, seeds or juice.

14. The composition as in claim 12, wherein the micro-organism Bacillus coagulans strain is preferably Bacillus coagulans MTCC 5856.

15. The composition as in claim 12, wherein the plant is preferably Cranberry.

16. The composition as in claim 12, wherein the cranberry species is selected from the group consisting of Vaccinium oxycoccos, Vaccinium microcarpum, Vaccinium macrocarpon, and Vaccinium erythrocarpum.

17. The composition as in claim 12, wherein the composition is formulated with pharmaceutically/nutraceutically acceptable excipients, adjuvants, bases, diluents, carriers, conditioning agents, bioavailability enhancers, antioxidants and preservatives and administered orally in form of tablets, capsules, syrups, gummies, powders, suspensions, emulsions, chewables, candies and eatables.

18. The composition as in claim 12, wherein composition is formulated with pharmaceutically/cosmeceutically acceptable excipients, adjuvants, bases, diluents, earners, conditioning agents, bioavailability enhancers, antioxidants and preservatives and/or incorporated into formulations containing skin care ingredients and administered topically in the form of creams, gels, lotions, powder, serum, oil, suspensions and ointments.
